(19) United States
(12) Patent Application Publication
(10) Pub. No.: US 2003/0068632 A1
(43) Pub. Date: Apr. 10, 2003

(54) Diagnosis of glaucoma

(75) Inventor: Henri-Jean Garchon, Paris (FR)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Institute National de la Sante et de la Recherche Medicale (INSERM)

(21) Appl. No.: 10/150,510

(22) Filed: May 16, 2002

Related U.S. Application Data
(63) Continuation-in-part of application No. 09/999,031, filed on Nov. 1, 2001.
Application No. 09/999,031 is a continuation of application No. PCT/US00/12179, filed on May 4, 2000.
(60) Provisional application No. 60/133,224, filed on May 7, 1999.

(51) Int. Cl. C12Q 1/68

(52) U.S. Cl. 435/6

(57) ABSTRACT

Methods are described for assessing an individual's risk for developing early-onset glaucoma, for developing glaucoma with a high intraocular pressure at onset of disease, or for developing glaucoma with a high visual field score and/or a high cup/disk ratio, by assessing the ApoE alleles and/or the ApoE gene promoter alleles of the individual. In individuals carrying mutations in the TIGR gene, the presence of an ApoE4 allele is indicative of an increased risk of developing early-onset glaucoma. In individuals carrying mutations in the TIGR gene promoter, the presence of an ApoE4 allele is indicative of a decreased risk of developing glaucoma with a high intraocular pressure at onset of disease. The combination of an ApoE4 allele and a “T” allele of a ApoE gene promoter (at −491) in an individual carrying a mutation in the TIGR gene is also indicative of an increased risk of developing early-onset glaucoma. The presence of a “T” allele of an ApoE gene promoter at (−491), regardless of whether a mutation in the TIGR gene is present or absent, is indicative of an increased risk of developing glaucoma with a high intraocular pressure at onset of disease. The presence of an ApoE(−219G) gene promoter allele is indicative of an increased risk of developing glaucoma with a high visual field score, high cup/disk ratio, or both.

### RELATED APPLICATIONS

This application is a continuation-in-part of U.S. application Ser. No. 09/999,031, filed Nov. 1, 2001, which is a continuation of International Application No. PCT/US00/12179, which designated the United States and was filed on May 4, 2000, published in English, which claims the benefit of U.S. Provisional Application No. 60/133,224, filed May 7, 1999. The entire teachings of the aforementioned applications are incorporated herein by reference.

### BACKGROUND OF THE INVENTION

Glaucoma is a group of ocular disorders, characterized by degeneration of the optic nerve. It is one of the leading causes of blindness worldwide. One major risk factor for developing glaucoma is family history: several different inherited forms of glaucoma have been described. One form of glaucoma, primary open angle glaucoma (gene symbol: GLC1), is a common disorder characterized by atrophy of the optic nerve resulting in visual field loss and eventual blindness. GLC1 has been divided into groups, based on age of onset and differences in clinical presentation.

Juvenile-onset primary open angle glaucoma (GLC1A) usually manifests in late childhood or early adulthood. The progression of GLC1A is rapid and severe with high intraocular pressure, is poorly responsive to medical treatment, and is such that it usually requires ocular surgery. GLC1A was initially mapped to the q21-q31 region of chromosome 1 (Sheffield, V. C. et al., Nature Genet. 4:47-50 (1993)); mutations in the gene for trabecular meshwork inducible glucocorticoid response (TIGR) protein, located a chromosome 1q24, have been identified as associated with GLC1A glaucoma (Stone, E. M. et al., Science 275:668-670 (1997); Stoilova, D. et al., Ophthalmic Genetics 18(3):109-118 (1997); Adam, M. F. et al., Hum. Mol. Genet. 6:2091-2097 (1997); Michels-Rautenstrauss, K. G., et al., Hum. Genet. 102:103-106 (1998); Mansergh, F. C. et al., Hum. Mutat. 11:244-251 (1998)).

Adult- or late-onset primary open angle glaucoma (GLC1B) is the most common type of glaucoma. It is milder and develops more gradually than juvenile-onset primary open angle glaucoma, with variable onset usually after the age of 40. GLC1B is associated with slight to moderate elevation of intraocular pressure, and often responds satisfactorily to regularly monitored medical treatment. However, because the disease progresses gradually and painlessly, it may not be detected until a late stage when irreversible damage to the optic nerve has already occurred. Linkage, haplotype and clinical data have assigned a locus for GLC1B to the 2cen-q13 region (Stoilova, D. et al., Genomics 36:142-150 (1996)).

Further evidence has identified several additional loci for primary open angle glaucoma. GLC1C, an adult-onset POAG gene, has been mapped to 3q (Wirtz, M. K. et al., am. J. Hum. Genet. 60:296-304 (1997)); GLC1D has been mapped to 8q23 (Trifan, O. C. et al., Am. J. Ophthalmol. 126:17-28 (1998)); GLC1E has been mapped to 10p15-p14 (Sarfarazi, M. et al., Am. J. Hum. Genet. 62: 641-652 (1998)).

Because of the insidious nature of glaucoma, a need remains for a better and earlier means to diagnose or predict the likelihood of development of glaucoma, so that preventative or palliative measures can be taken before significant damage to the optical nerve occurs.

### SUMMARY OF THE INVENTION

The invention pertains to methods of assessing risk for developing early-onset glaucoma, and to methods of assessing risk for developing glaucoma with a high intraocular pressure (IOP) at the onset of disease, in an individual, such as an individual who has a mutation in the gene for trabecular meshwork inducible glucocorticoid response (TIGR) protein (a “carrier of a TIGR gene mutation”) or an individual who has a mutation in the promoter of the TIGR gene (a “carrier of a TIGR gene promoter mutation”). The invention also pertains to kits useful in the methods.

The methods comprise assessing the individual's alleles of the apolipoprotein E (ApoE) gene, and/or assessing the individual's alleles of the promoter of an ApoE gene, in order to determine whether the individual has an ApoE4 allele (or two ApoE4 alleles); and/or whether the individual has a “T” allele (or two “T” alleles) of an ApoE gene promoter at (−491) (e.g., by detection of the presence or absence of ApoE4 allele(s), and/or by detection of the presence or absence of “T” allele(s) of an ApoE gene promoter); and/or whether the individual has an ApoE(−219G) gene promoter allele. If it is not known whether the individual is a carrier of a TIGR gene mutation or a TIGR gene promoter mutation, the presence or absence of a mutation in the TIGR gene or promoter can be determined concurrently with the assessment of the ApoE alleles and/or the ApoE gene promoter alleles.

In a carrier of a TIGR gene mutation, the presence of an ApoE4 allele is indicative of an increased risk of developing early-onset glaucoma, compared with the risk of a carrier of a TIGR gene mutation with no ApoE4 alleles. The presence of an ApoE4 allele in a carrier of a TIGR gene promoter mutation is indicative of a decreased risk of developing glaucoma with a high intraocular pressure at onset of disease, compared with the risk of a carrier of a TIGR gene promoter mutation with no ApoE4 alleles. The absence of any ApoE4 alleles in a carrier of a TIGR gene mutation is indicative of a decreased risk of developing early-onset glaucoma, compared with the risk of a carrier of a TIGR gene mutation with an ApoE4 allele. The absence of any ApoE4 alleles in a carrier of a TIGR gene promoter mutation is also indicative of an increased risk of developing glaucoma with a high intraocular pressure at onset of disease, compared with the risk of a carrier of a TIGR gene promoter mutation with an ApoE4 allele.

The combination of an ApoE4 allele and a “T” allele of a ApoE gene promoter (at −491) in an individual carrying a mutation in the TIGR gene is also indicative of an increased risk of developing early-onset glaucoma, compared with the risk of a carrier of a TIGR mutation with an ApoE4 allele but no “T” alleles of a ApoE gene promoter. The absence of any “T” alleles of an ApoE gene promoter in a carrier of a TIGR mutation with an ApoE4 allele is indicative of a decreased risk of developing early-onset glaucoma, compared with the risk of a carrier of a TIGR mutation with an ApoE4 allele and a “T” allele of an ApoE gene promoter.

The presence of a “T” allele of an ApoE gene promoter in an individual, regardless of whether a mutation in the TIGR gene is present or absent, is indicative of an increased risk of developing glaucoma with a high intraocular pressure at onset of disease, compared with the risk of an individual who has no “T” alleles of an ApoE gene promoter. The absence of a “T” allele of an ApoE gene promoter in an individual, is indicative of a decreased risk of developing glaucoma with a high intraocular pressure at onset of disease, compared with the risk of an individual who has a “T” allele of an ApoE gene promoter. Furthermore, if the individual is a carrier of a TIGR gene promoter mutation (e.g., a (−1000G) mutation), the presence of an ApoE(−491T) gene promoter allele is indicative of an even greater increased risk of developing glaucoma with a high intraocular pressure at onset of disease, compared with the risk of such an individual who has no ApoE(−491T) gene promoter allele or TIGR gene promoter mutation.

The presence of an ApoE(−219G) gene promoter allele is indicative of an increased risk of developing glaucoma with a high visual field score, a high cup/disk ratio, or both, compared with the risk of an individual who has no ApoE(−219G) gene promoter allele. The absence of an ApoE(−291G) gene promoter allele in an individual is indicative of a decreased risk of developing glaucoma with a high visual field score or a high cup/disk ratio, compared with the risk of an individual who has an ApoE(−219G) gene promoter allele.

The methods and kits of the invention afford a simple means to identify individuals at risk for early onset of glaucoma or for more severe glaucoma, as high intraocular pressure, particularly at the onset of the disease, is associated with more severe glaucoma. Identification of those at increased risk enables better treatment planning for affected individuals, as well as for other family members who may be affected individuals or disease gene carriers.

### DETAILED DESCRIPTION OF THE INVENTION

The invention relates to methods of assessing an individual's risk for developing early-onset glaucoma, and for assessing an individual's risk for developing glaucoma with a high intraocular pressure at onset of the disease. The term “glaucoma”, as used herein, refers to primary open angle glaucoma (POAG), including both juvenile-onset and adult- or late-onset POAG.

As described herein, Applicant has identified a relationship between alleles of the apolipoprotein E (ApoE) gene and the age of onset of glaucoma, as well as a relationship between alleles of an ApoE gene promoter and the level of intraocular pressure at diagnosis of glaucoma. Applicant has also identified a relationship between alleles of the ApoE gene promoter and the level of intraocular pressure at diagnosis of glaucoma, as well as a relationship between alleles of the ApoE gene promoter and the development of glaucoma with a high visual field score and/or a high cup/disk ratio.

Apolipoprotein E (ApoE) is a protein constituent of plasma lipoproteins and plays a role in cholesterol metabolism. There are three isoforms of ApoE protein, ApoE2, ApoE3 and ApoE4, which are produced by three ApoE alleles of a single gene. An individual may have one of six phenotypes, depending on which alleles the individual has: a homozygous phenotype (ApoE2/2, ApoE3/3, or ApoE4/4), or a heterozygous phenotype (ApoE2/3, ApoE2/4, or ApoE3/4 (see Mahley, R. W., Science 240:622-630 (1988); Emi, M. et al., Genomics 3:373-379 (1988); the teachings of these references are incorporated herein by reference in their entirety). A biallelic (A/T) polymorphism in the gene promoter for the ApoE gene has also been identified (at position −491; see Bullido, M. J., et al., Nature Genet. 18:69-71 (1998)), the teachings of which are incorporated herein by reference in their entirety.

Several mutations in the TIGR gene have been associated with glaucoma (see, e.g., Richards, J. E. et al., Ophthalmology 105:1698-1707 (1998); Kee, C. and Ahn, B. H., Korean J. Ophthalmol. 11:75-78 (1997); Adam, M. F. et al., Hum. Mol. Genet 6:2091-2097 (1997)). More than one polymorphism or mutation in the promoter for the TIGR gene have also been identified.

Applicant has identified a correlation between ApoE alleles and age of onset of glaucoma in individuals having one or more mutation(s) in the gene encoding trabecular meshwork inducible glucocorticoid response (TIGR) protein (the “TIGR gene”), particularly mutations with variable expressivity. Individuals having one or more mutation(s) in the gene encoding the TIGR protein are also referred to herein as “TIGR gene mutation carriers” or “TIGR protein mutation carriers,” to indicate that the mutation is in the region that encodes the TIGR protein; individuals having one or more mutation(s) in the promoter for the TIGR gene are referred to herein as “TIGR gene promoter mutation carriers.” An individual may be both a TIGR gene mutation carrier and a TIGR gene promoter mutation carrier.

In TIGR gene mutation carriers, those who have at least one ApoE4 allele were significantly younger at the time of onset of glaucoma, than individuals having no ApoE4 alleles. Those TIGR gene mutation carriers having no ApoE3 alleles and at least one ApoE2 allele, were significantly older at the time of onset of glaucoma, than TIGR gene mutation carriers having at least one ApoE4 allele. TIGR gene mutation carriers who were homozygous for ApoE3 allele had an age of onset of glaucoma that was intermediate between those having at least one ApoE4 allele and those having no ApoE4 alleles and at least one ApoE2 allele. In addition, in TIGR gene mutation carriers having at least one ApoE4 allele, the presence of one or two T allele(s) in the gene promoter of an ApoE gene is also associated with a significantly younger age of onset of glaucoma, compared to the presence of two A alleles. Furthermore, the T allele of the gene promoter affected the level of intraocular pressure (IOP) at the onset of disease, regardless of whether the individual was a TIGR gene mutation carrier or not: individuals who have a T allele in the gene promoter of any ApoE allele had a significantly higher IOP at diagnosis. The T allele polymorphism in the gene promoter was also associated with higher IOP in TIGR gene promoter mutation carriers. In contrast, the IOP was lower for those TIGR gene promoter mutation carriers having an ApoE4 allele. Individuals having an ApoE(−219G) gene promoter allele had a significantly higher visual field score and significantly higher cup/disk ratio (indications of greater optic nerve alteration) than individuals not having an ApoE(−219G) gene promoter allele. Furthermore, the visual field score and cup/disk ratio were even more greatly increased in individuals having an ApoE(−219G) gene promoter allele and a mutation in the TIGR promoter (−1000G).

As a result of these discoveries, methods of assessing an individual's risk for developing early-onset glaucoma, as well as methods of assessing an individual's risk for developing glaucoma with a high intraocular pressure at onset of disease, and also methods of assessing an individual's risk for developing a glaucoma with a high visual field score and/or high cup/disk ratio, are now available. In the methods, the ApoE alleles in the individual, and/or the alleles of the ApoE gene promoter, are assessed.

To assess the ApoE alleles in the individual, the presence or absence of one or more particular ApoE alleles are detected. The presence of an ApoE2, ApoE3, or ApoE4 allele, or a combination thereof, can be detected, provided that the detection of the presence or absence of particular ApoE alleles is conducted so that it can be determined whether an individual has at least one ApoE4 allele. For example, the presence or absence of an ApoE4 allele (or two ApoE4 alleles, if two are present) can be detected. Alternatively, because the presence of two alleles also indicates the absence of any other allele, the presence or absence of ApoE2 and/or ApoE3 alleles can be detected, thereby providing an indirect assessment of the presence or absence of any ApoE4 allele(s). For example, the presence of two ApoE3 alleles (the most common genotype), of two ApoE2 alleles, or of one ApoE3 and one ApoE2 allele, is indicative of the absence of any ApoE4 alleles. If desired, the presence or absence of all three alleles (ApoE2, ApoE3 and ApoE4) can be detected.

To assess the alleles of the ApoE gene promoter, the presence or absence of an ApoE gene promoter allele is detected. For example, in one embodiment, the presence or absence of the “T” allele of an ApoE gene promoter (at −491) can be detected; alternatively, the presence or absence of the “A” allele of an ApoE gene promoter can be detected, thereby providing an indirect assessment of the presence or absence of a “T” allele. If desired, the presence or absence of both alleles (the “T” allele and the “A” allele) can be detected. In another embodiment, the presence or absence of the (−219G) gene promoter allele can be detected; alternatively, the presence or absence of a (−491A) gene promoter allele or a (−219C) gene promoter allele can be detected, thereby providing an indirect assessment of the presence or absence of the (−491T) gene promoter allele or the (−219G) gene promoter allele. If desired, the presence or absence of both alleles (e.g., −491T and −491A, or −219G and −219C), can be detected. In another embodiment, more than one of the different promoter alleles described herein (e.g. −491 and −219) can be detected concurrently.

The ApoE alleles and/or the ApoE gene promoter alleles in an individual can be assessed by a variety of methods, including hybridization methods (e.g., Southern or Northern analysis), sequencing of the gene and/or the gene promoter, allele-specific oligonucleotide analysis, analysis by restriction enzyme digestion, or (in the case of the ApoE alleles) by analysis of the ApoE protein(s) (e.g., spectroscopy, enzyme-linked immunosorbent assay, colorimetry, electrophoresis, isoelectric focusing, radioimmunoassay, immunoblotting (such as Western blotting)). Several methods of assessing the ApoE alleles are described in detail in U.S. Pat. No. 5,508,167 to Roses et al., the entire teachings of which are incorporated herein by reference. Similar methods can be used to assess the alleles of the ApoE promoter.

For example, in one method of assessing the ApoE alleles in the individual, hybridization methods, such as Southern analysis, can be used (see Current Protocols in Molecular Biology, Ausubel, F. et al., eds., John Wiley & Sons, including all supplements through 1998). For example, a test sample containing genomic DNA, RNA, or cDNA that includes the ApoE gene or encodes ApoE protein can be used. Such genomic DNA, RNA and cDNA are referred to herein collectively as “nucleic acids comprising the ApoE gene”. The test sample is obtained from an individual (the “test individual”). The individual can be an adult, child, or fetus. The test sample can be from any source which contains DNA, RNA or cDNA, such as a blood sample, cerebrospinal fluid sample, or tissue sample (e.g., from skin or other organs). In a preferred embodiment, a test sample containing nucleic acids comprising ApoE gene is obtained from a blood sample, a fibroblast skin sample, from hair roots, or from cells obtained from the oral cavity (e.g., via mouthwash). In another preferred embodiment, a test sample containing nucleic acids comprising the ApoE gene is obtained from fetal cells or tissue by appropriate methods, such as by amniocentesis or chorionic villus sampling.

If desired, the ApoE gene can be amplified, such as by polymerase chain reaction (PCR) (see, e.g., U.S. Pat. Nos. 4,683,195, 4,683,202, 4,800,159, 4,965,188), ligase chain reaction (LCR) (see, e.g., Weiss, R., Science 254:1292 (1991)), or other means. Alternatively, a portion of the ApoE gene can be amplified (e.g., a portion including codon 112 or codon 158). The test sample containing the nucleic acids comprising the ApoE gene (and amplified copies of the gene or portion of the gene, if amplification is performed) is then examined to assess the ApoE alleles. The presence of a particular allele is indicated by hybridization of nucleic acids comprising the ApoE gene in the test sample to a nucleic acid probe. A “nucleic acid probe”, as used herein, can be a DNA probe or an RNA probe. The nucleic acid probe specifically hybridizes to only one of the alleles of the ApoE gene; that is, it hybridizes to one allele (e.g., to the ApoE4 allele), but not to either of the other alleles (e.g., the ApoE2 and ApoE3 alleles). Such a nucleic acid probe is referred to herein as an “allele-specific nucleic acid probe.” A fragment of such a nucleic acid probe can also be used, provided that the fragment hybridizes to the part of the ApoE gene that contains the allelic variation.

To assess the ApoE alleles, a hybridization sample is formed by contacting the test sample containing the nucleic acid comprising the ApoE gene, with at least one nucleic acid probe. The hybridization sample is maintained under conditions which are sufficient to allow specific hybridization of the nucleic acid probe to the nucleic acid comprising the ApoE gene. “Specific hybridization”, as used herein, indicates exact hybridization (e.g., with no mismatches). Specific hybridization can be performed under high stringency conditions or moderate stringency conditions, for example. “Stringency conditions” for hybridization is a term of art which refers to the conditions of temperature and buffer concentration which permit hybridization of a particular nucleic acid to another nucleic acid in which the first nucleic acid may be perfectly complementary to the second, or the first and second nucleic acids may share only some degree of complementarity. For example, certain high stringency conditions can be used which distinguish perfectly complementary nucleic acids from those of less complementarity. “High stringency conditions” and “moderate stringency conditions” for nucleic acid hybridizations are explained in chapter 2.10 and 6.3, particularly on pages 2.10.1-2.10.16 and pages 6.3.1-6 in Current Protocols in Molecular Biology, supra, the teachings of which are hereby incorporated by reference. The exact conditions which determine the stringency of hybridization depend on factors such as length of nucleic acids, base composition, percent and distribution of mismatch between the hybridizing sequences, temperature, ionic strength, concentration of destabilizing agents, and other factors. Thus, high or moderate stringency conditions can be determined empirically. In one embodiment, the hybridization conditions for specific hybridization are moderate stringency. In a particularly preferred embodiment, the hybridization conditions for specific hybridization are high stringency.

Specific hybridization, if present, is then detected using standard methods. If specific hybridization occurs between the allele-specific nucleic acid probe and an ApoE gene in the test sample, then the individual has the allele of ApoE to which that nucleic acid probe hybridizes. More than one nucleic acid probe can also be used concurrently in this method (e.g., a probe that hybridizes to an ApoE2 allele and a probe that hybridizes to an ApoE3 allele).

Similar methods can also be used to assess the ApoE gene promoter alleles, using a sample which contains nucleic acids of the gene promoter (and amplified copies of the gene promoter or portion of the gene promoter, if amplification is performed) and allele-specific nucleic acid probes that hybridize to only one of the two ApoE gene promoter alleles. In addition, these methods can be used to assess both the ApoE alleles and the ApoE gene promoter alleles concurrently, using a sample which contains nucleic acids comprising the ApoE gene and also comprising the ApoE gene promoters (and amplified copies of the gene and gene promoter, or portions of the gene or gene promoter, if amplification is performed), at least one allele-specific nucleic acid probe that hybridizes to one of the ApoE alleles, and an allele-specific nucleic acid probe that hybridizes to an allele of the ApoE promoter. For example, genomic DNA comprising the ApoE promoter and the ApoE gene can be amplified concurrently and then assessed for the alleles of the gene and the promoter.

In another hybridization method, Northern analysis (see Current Protocols in Molecular Biology, Ausubel, F. et al., eds., John Wiley & Sons, supra) is used to identify the presence or absence of an allele of the ApoE gene. For Northern analysis, a sample of RNA is obtained from the test individual by appropriate means. Specific hybridization of an allele-specific nucleic acid probe, as described above, to RNA from the individual is indicative of the presence of that allele of the ApoE gene. For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry, 1994, 5, American Chemical Society, p. 1 (1994). The PNA probe can be designed in a similar manner as the nucleic acid probes described above, that is, to specifically hybridize to a particular allele of the ApoE gene.

Sequence analysis can also be used to detect the alleles of the ApoE gene and/or the alleles of the ApoE gene promoter. A test sample is obtained from the test individual, as described above. As described above, PCR or LCR can be used to amplify the gene, gene promoter, and/or its flanking sequences, if desired. The sequence(s) of the alleles of the ApoE gene, or a fragment of the gene, and/or the sequence(s) of the ApoE gene promoter, is determined, using standard methods. The sequence(s) of the ApoE gene, gene fragment, or gene promoter is compared with the known nucleic acid sequences of the different alleles of the ApoE gene or ApoE gene promoter, and the alleles of the individual are thereby determined.

Allele-specific oligonucleotides (also referred to herein as “sequence-specific oligonucleotides”) can also be used to detect the presence or absence of ApoE alleles and/or ApoE gene promoter alleles, through the use of dot-blot hybridization of amplified nucleic acids with allele-specific oligonucleotide (ASO) probes (see, for example, Houlston, R. S. et al., Hum. Genet. 83:364-8 (1989), the entire teachings of which are incorporated herein by reference). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to one allele of the ApoE gene (or to one allele of the ApoE gene promoter, depending on whether the ApoE alleles or the ApoE gene promoter alleles are being assessed). An allele-specific oligonucleotide probe that is specific for particular alleles of the gene or of the gene promoter can be prepared, using standard methods (see Current Protocols in Molecular Biology, supra). To determine the alleles of the ApoE gene or gene promoter, a test sample is obtained from the test individual as described above. PCR or LCR can be used to amplify all or a fragment of the ApoE gene, gene promoter, and/or its flanking sequences, if desired. The test sample is dot-blotted, using standard methods (see Current Protocols in Molecular Biology, supra), and the blot is contacted with the allele-specific oligonucleotide probe(s). The presence of specific hybridization of one or more probes to the test sample is then detected. Specific hybridization of an allele-specific oligonucleotide probe to the test sample of the individual is indicative of the presence of that allele of the ApoE gene (or gene promoter) to which the allele-specific oligonucleotide binds.

Assessment of the ApoE alleles can also be made by examining a test sample comprising ApoE protein. A test sample from an individual is assessed for the presence of protein encoded by one or more alleles of the ApoE gene. Various means of examining protein encoded by the ApoE gene can be used, including spectroscopy, enzyme-linked immunosorbent assay (ELISA), colorimetry, electrophoresis, isoelectric focusing, and immunoblotting (see Current Protocols in Molecular Biology, particularly chapter 10). For example, Western blotting analysis, using an antibody that specifically binds to a protein encoded by one allele of the ApoE gene, can be used to identify the presence or absence in a test sample of a protein encoded by that allele of the ApoE gene. The term “antibody”, as used herein, encompasses both polyclonal and monoclonal antibodies, as well as mixtures of more than one antibody reactive with the protein or protein fragment (e.g., a cocktail of different types of monoclonal antibodies reactive with the mutant protein or protein fragment). The term antibody is further intended to encompass whole antibodies and/or biologically functional fragments thereof, chimeric antibodies comprising portions from more than one species, humanized antibodies, human-like antibodies, and bifunctional antibodies. Biologically functional antibody fragments are those fragments sufficient for binding of the antibody fragment to the protein of interest.

In preferred embodiments of the invention, the ApoE alleles and/or ApoE gene promoter alleles are assessed using dot-blot hybridization with SSO probes, preferably after allele-specific amplification of the relevant genetic material (i.e., amplification of the ApoE gene or ApoE gene promoter).

### METHODS OF THE INVENTION

In one embodiment of the invention, an individual's risk for developing early-onset glaucoma is assessed. The individual can be an individual who is known to be a TIGR gene mutation carrier and/or a TIGR gene promoter mutation carrier; alternatively, the individual's status as a TIGR gene mutation carrier and/or TIGR gene promoter mutation carrier (whether or not the individual is a TIGR gene mutation carrier or a TIGR gene promoter mutation carrier) can be unknown, and can be determined at a different time. In another embodiment, the individual's status as a TIGR gene mutation carrier and/or a TIGR gene promoter mutation carrier can be determined concurrently, using methods similar to those described above to identify mutation(s) in the TIGR gene (see, e.g., Richards, J. E. et al., Ophthalmology 105:1698-1707 (1998); and Kee, C. and Ahn, B. H., Korean J. Ophthalmol. 11:75-78 (1997); the entire teachings of these references are incorporated herein by reference). In a preferred embodiment, the individual's status as a TIGR gene mutation carrier and/or a TIGR gene promoter mutation carrier is determined concurrently, by detecting a mutation in the TIGR gene and/or in the TIGR gene promoter.

To assess an individual's risk for developing early-onset glaucoma, the ApoE alleles of the individual are assessed, in order to determine (directly or indirectly) whether the individual has an ApoE4 allele (i.e., whether the individual has at least one ApoE4 allele; the individual may also have two ApoE4 alleles), as described above. In a preferred embodiment, the ApoE alleles of the individual are assessed by determining the presence or absence of an ApoE4 allele (e.g., determining the absence of any ApoE4 alleles; the presence of one ApoE4 allele; ir the presence of two ApoE4 alleles). If the individual is a TIGR gene mutation carrier, and the individual has an ApoE4 allele (that is, has at least one ApoE4 allele; the individual may also have two ApoE4 alleles), then the individual has an increased risk of developing early-onset glaucoma, compared to a TIGR gene mutation carrier having no ApoE4 alleles. Such an individual who has “increased risk of developing early-onset glaucoma” is an individual who is likely to have an age of onset of glaucoma which is younger, by an amount that is statistically significant, than the age of onset of glaucoma for a TIGR gene mutation carrier having no ApoE4 alleles. If a TIGR gene mutation carrier has no ApoE4 alleles, that individual has a decreased risk of developing early-onset glaucoma, compared to a TIGR gene mutation carrier having an ApoE4 allele (or two ApoE4 alleles); that is, is likely to have an age of onset of glaucoma which is older, by an amount that is statistically significant, than the age of onset of glaucoma for a TIGR gene mutation carrier having one or two ApoE4 allele(s). Furthermore, a TIGR gene mutation carrier having one ApoE2 and one ApoE3 allele, or two ApoE2 alleles, has a decreased risk of developing early-onset glaucoma (i.e., is likely to have an age of onset of glaucoma which is older, by an amount that is statistically significant), compared to a TIGR gene mutation carrier having two ApoE3 alleles.

In another embodiment of the invention, an individual's risk for developing early-onset glaucoma is assessed by assessing the ApoE promoter alleles of the individual, in order to determine (directly or indirectly) whether the individual has a “T” allele (or two “T” alleles) in an ApoE gene promoter, as described above. In a preferred embodiment, the ApoE gene promoter alleles of the individual are assessed by determining the presence or absence of a “T” allele of an ApoE gene promoter (e.g., the absence of any “T” alleles; the presence of one “T” allele; or the presence of two “T” alleles). As above, the individual can be an individual who is known to be a TIGR mutation carrier; alternatively, the individual's status as a TIGR mutation carrier (whether or not the individual is a TIGR mutation carrier) can be unknown, and can be determined at a different time, or can be determined concurrently. In addition, the individual can be an individual who is known to have at least one ApoE4 allele; alternatively, the individual's ApoE allele status can be unknown and can be determined at a different time or concurrently. In a preferred embodiment, the individual's ApoE gene promoter alleles are assessed concurrently with the individual's ApoE alleles.

If the individual is a TIGR gene mutation carrier and has an ApoE4 allele(s), and also has a “T” allele of an ApoE gene promoter (that is, has at least one “T” allele; the individual may also have two “T” alleles), then the individual has an increased risk of developing early-onset glaucoma. Such an individual who has an increased risk of developing early-onset glaucoma is an individual who is likely to have an age of onset of glaucoma which is younger, by an amount that is statistically significant, compared to a TIGR gene mutation carrier having an ApoE4 allele(s) but having no “T” alleles of an ApoE gene promoter. If a TIGR gene mutation carrier having an ApoE4 allele(s) has no “T” alleles of an ApoE gene promoter, that individual has a decreased risk of developing early-onset glaucoma, compared to a TIGR gene mutation carrier having an ApoE4 allele(s) and no “T” alleles of an ApoE gene promoter (that is, is likely to have an age of onset of glaucoma which is older, by an amount that is statistically significant, than the age of onset of glaucoma for a TIGR gene mutation carrier having an ApoE4 allele(s) and a “T” allele(s) of an ApoE gene promoter).

In another embodiment of the invention, an individual's risk for developing glaucoma with a high intraocular pressure (IOP) at onset of disease is assessed. “Onset of disease” indicates the time when symptoms of glaucoma are first exhibited. While a precise moment of onset of disease may not be determinable, a high IOP at initial diagnosis of glaucoma is indicative of a high IOP at onset of disease.

The individual can be an individual who is known to be a TIGR gene mutation carrier and/or a TIGR gene promoter mutation carrier; alternatively, the individual's status as a TIGR gene mutation carrier and/or a TIGR gene promoter mutation carrier (whether or not the individual is a TIGR gene mutation carrier and/or a TIGR gene promoter mutation carrier) can be unknown. To assess the individual's risk for developing glaucoma with a high IOP at onset of disease, the ApoE promoter alleles of the individual are assessed, in order to determine (directly or indirectly) whether the individual has a “T” allele(s) in the ApoE promoter (at −491), as described above. In a preferred embodiment, the ApoE gene promoter alleles of the individual are assessed by determining the presence or absence of a “T” allele (e.g., the absence of any “T” alleles; the presence of one “T” allele; or the presence of two “T” alleles) of an ApoE gene promoter. If the individual has a “T” allele (or two “T” alleles) of an ApoE gene promoter, then the individual has an increased risk of developing glaucoma with a high IOP at onset of disease, compared to an individual having no “T” alleles of an ApoE gene promoter. An individual who has “increased risk of developing glaucoma with a high IOP at onset of disease” is an individual who is likely to have an IOP at onset of disease that is higher, by an amount that is statistically significant, than the IOP at onset of disease for an individual having no “T” alleles of an ApoE gene promoter. If an individual has no “T” alleles of an ApoE gene promoter, that individual has a decreased risk of developing glaucoma with a high IOP at onset of disease, compared to an individual having a “T” allele (or two “T” alleles) of an ApoE gene promoter (that is, is likely to have an IOP at onset of disease which is lower, by an amount that is statistically significant, than the IOP at onset of disease for an individual having a “T” allele(s) of an ApoE gene promoter.

Furthermore, if the individual is a TIGR gene promoter mutation carrier (e.g., having (−1000G)), and the individual has one (or more) ApoE(−491T) gene promoter allele(s), then the individual has an even more increased risk of developing glaucoma with a high IOP at onset of disease (that is, the individual is likely to have an IOP at onset of disease which is higher, by an amount that is statistically significant), compared to an individual not having any ApoE(−491T) gene promoter alleles or a TIGR gene promoter mutation.

In another embodiment of the invention, an individual's risk for developing glaucoma with a high IOP at onset of disease is assessed by assessing the ApoE alleles of the individual, in order to determine (directly or indirectly) whether the individual has an ApoE4 allele(s), as described above. In a preferred embodiment, the ApoE alleles of the individual are assessed by determining the presence or absence of an ApoE4 allele(s). The individual can be an individual who is known to be a TIGR gene mutation carrier and/or a TIGR gene promoter mutation carrier; alternatively, the individual's status as a TIGR gene mutation carrier and/or a TIGR gene promoter mutation carrier (whether or not the individual is a TIGR gene mutation carrier and/or a TIGR gene promoter mutation carrier) can be unknown, and can be determined at a different time, or can be determined concurrently. In a preferred embodiment, the individual's status as a TIGR gene mutation carrier and/or a TIGR gene promoter mutation carrier is determined concurrently using methods as described above.

If the individual is a TIGR gene promoter mutation carrier an ApoE4 allele (or two ApoE4 alleles), then the individual has a decreased risk of developing glaucoma with a high IOP at onset of disease (that is, the individual is likely to have an IOP at onset of disease which is lower, by an amount that is statistically significant), compared to a TIGR gene promoter mutation carrier having ApoE4 alleles. If a TIGR gene promoter mutation carrier has no ApoE4 alleles, that individual has an increased risk of developing glaucoma with a high IOP at onset of disease, compared to a TIGR gene promoter mutation carrier having an ApoE4 allele(s) (that is, is likely to have an IOP at onset of disease which is higher, by an amount that is statistically significant, than the IOP at onset of disease for a TIGR gene promoter mutation carrier having one or two ApoE4 alleles).

In another embodiment of the invention, an individual's risk for developing glaucoma with a high visual field score, a high cup/disk ratio, or both, is assessed by assessing the ApoE gene promoter alleles of the individual, in order to determine (directly or indirectly) whether the individual has an ApoE(−219G) gene promoter allele(s), as described above. If the individual has one or more ApoE(−219G) gene promoter allele(s), then the individual has an increased risk of developing glaucoma with a high visual field score, a high cup/disk ratio, or both (that is, the individual is likely to have a visual field score, a cup/disk ratio, or both, that is higher by an amount that is statistically significant), compared to an individual not having any ApoE(−219G) gene promoter alleles. If an individual has no ApoE(−219G) gene promoter alleles, that individual has a decreased risk of developing glaucoma with a with a high visual field score or a high cup/disk ratio, compared to an individual having an ApoE(−219G) gene promoter allele(s) (that is, is likely to have a visual field score, a cup/disk ratio, or both, that is lower, by an amount that is statistically significant, than the visual field score, cup/disk ratio, or both, of an individual carrier having one or two ApoE(−219G) gene promoter alleles).

### KITS OF THE INVENTION

The present invention also includes kits useful in the methods of the invention. The kits can include a means for obtaining a test sample; nucleic acid probes, PNA probes, or allele-specific oligonucleotide probes; appropriate reagents; antibodies to ApoE isoforms; instructions for performing the methods of the invention; control samples; and/or other components. In a preferred embodiment, the kit includes a means for assessing the ApoE alleles and the alleles of the ApoE gene promoter of an individual, and also instructions for performing the methods of the invention. In another preferred embodiment, the kit includes a means for assessing the status of an individual as a TIGR gene mutation carrier and/or a TIGR gene promoter mutation carrier, as well as a means for assessing the ApoE alleles and the alleles of the ApoE gene promoter of the individual, and also instructions for performing the methods of the invention.

The teachings of all references cited herein are incorporated by reference in their entirety. The invention is further illustrated by the following Examples.

### EXAMPLE 1

### Identification of Relationship Between ApoE Alleles, ApoE Gene Promoter Alleles, and Glaucoma

### Assesment of ApoE Alleles and ApoE Gene Promoter Alleles

Patients were selected as described in Adam, M. F. et al. (Hum. Mol. Genet. 6:2091-2097 (1997)). Samples were taken and genomic DNA was prepared as described by Sambrook et al. (Molecular Cloning: A Laboratory Manual, 2^{nd} ed., New York: Cold Spring Harbor Laboratory Press, 1989).

Assessment of the ApoE alleles was performed by genotyping of the ApoE coding region polymorphisms. Polymerase chain reaction (PCR), followed by dot-blot hybridization with sequence-specific oligonucleotide (SSO) probes was utilized. The ApoE gene was amplified between bases 3878 and 4207 with the following primers:

| | |
|---|---|
| forward, TCCAAGGAGCTGCAGGCGGCGCA; | (SEQ ID NO:1) |
| reverse, TAGCGGCTGGCCGGCCAGGGAG. | (SEQ ID NO:2) |

The reaction included 400 ng genomic DNA, 12 pmoles of the forward primer, 16 pmoles of the reverse primer, 0.7 units of Taq DNA polymerase (Promega, Madison, Wis.), 2 mM MgCl₂, 10% v/v DMSO, 200 μM each dNTP, plus 1× enzyme buffer (Promega), for a final volume of 50 μl. Amplification was carried out in a PHC3 thermal cycler (Techne, UK), during 44 cycles of 3 segments each (1 minute at 94° C., I minute at 60° C., 1 minute at 72° C.). PCR products were dot-blotted on Hybond N+nylon membrane (Amersham, UK), then denatured in NaOH 0.4 M. The polymorphic codons were probed with 4 oligonucleotides radiolabled with ATP[γ³²P] using the T4 kinase (Promega). The sequences of the probes were as follows (polymorphic positions are underlined):

Codon 112: T: GCACACGTCCTCCATG (SEQ ID NO: 3) (wash temp 50° C.);

Codon 112: G: CATGGAGGACGTGCGC (SEQ ID NO: 4) (wash temp 50° C.);

Codon 158: T: GCACTTCTGCAGGTCA (SEQ ID NO: 5) (wash temp 48° C.); and

Codon 158: G: TGACCTGCAGAAGCGC (SEQ ID NO: 6) (wash temp 50° C.).

Hybridization was performed overnight at 42° C. in 5× SSPE, 0.1% SDS, 1% nonfat dry milk (20× SSPE is 3 M NaCl, 20 mM EDTA, 0.1 M sodium phosphate pH 7.4). Excess probe was washed away with wash solution (4× SSPE, 0.1% SDS) at the indicated temperature. Membranes were autoradiographed to a XAR5 Kodak film. To control for the amount of blotted PCR product, membranes were dehybridized and reprobed with the radiolabled forward amplification primer. Combinations of polymorphic positions determine the 3 known alleles of ApoE:

112 T+158 T=ApoE2

112 T+158 G=ApoE3

158 G+158 G=ApoE4.

Assessment of the ApoE gene promoter alleles was performed by analysis of the ApoE gene promoter region polymorphisms. Polymerase chain reaction (PCR), followed by dot-blot hybridization with sequence-specific oligonucleotide (SSO) probes was utilized. Amplification was performed with the following primers:

| | |
|---|---|
| Forward: GTGCATCATACTGTTCCCAC, and | (SEQ ID NO:7) |
| Reverse: TCCTTTCCTGACCCTGTCCTT. | (SEQ ID NO:8) |

The reaction included 200 ng genomic DNA, 5 pmoles of each primer, 0. 125 units of Taq DNA polymerase (Promega, Madison, Wis.), 1.5 mM MgCl₂, 200 μM each dNTP, plus 1× enzyme buffer (Promega), for a final volume of 25 μl. Amplification was carried out in a PHC3 thermal cycler (Techne, UK), during 35 cycles of 3 segments each (1 minute at 94° C., 1 minute at 53° C., 1 minute at 72° C.). A second allele-specific amplification was performed with the forward primer of the first PCR step , and one of the two allele-specific primers: AATCACTTAAGGTCAGGAG[T/A] (SEQ ID NO: 9, 10). The PCR products were separated by migration on an agarose gel and visualized by ethidium bromide staining and UV transillumination.

### Relationship Between ApoE Alleles, ApoE Promoter Alleles, and Glaucoma Phenotypes (Age of Onset and Initial IOP)

The age at disease onset of patients with glaucoma caused by a mutation of the TIGR protein was assessed. Survival analysis indicated that carriers of the TIGR mutation who had at least one ApoE4 allele, were significantly younger (p=0.01) at the time of diagnosis than patients with no ApoE4 allele and with at least one ApoE2 allele; those homozygous for the ApoE3 allele had an intermediate age of onset. The ApoE gene promoter polymorphism by itself was not influential. However, in individuals with an ApoE4 allele, the “T” allele of the ApoE gene promoter (approximately 15% of controls) was associated with a younger age of onset than the “A” allele (comparison of AA versus (AT=TT) in ApoE4 individuals). Thus, patients with both ApoE4 and ApoE “T” gene promoter alleles had a much younger age of onset (<30 years) than non-ApoE4 patients. The data are provided in Tables 1 and 2 below.

TABLE 1
Life Table for Patients with TIGR Gene Mutation and ApoE Alleles

| | | | | |
|---|---|---|---|---|
| | | No. | | |
| | No. | with | | Cum. % |
| No. Enter | Censored | Disease | Surviving | Surviving |

| ApoE: | 2/3 | 3/4 | 2/3 | 3/4 | 2/3 | 3/4 | 2/3 | 3/4 | 2/3 | 3/4 |
|---|---|---|---|---|---|---|---|---|---|---|
| Age | | | | | | | | | | |
| 8.00 | 4 | 20 | 0 | 3 | 0 | 2 | 100.0 | 89.2 | 100.0 | 100.0 |
| 13.78 | 4 | 15 | 1 | 1 | 0 | 2 | 100.0 | 86.2 | 100.0 | 89.2 |
| 19.56 | 3 | 12 | 0 | 0 | 0 | 1 | 100.0 | 91.7 | 100.0 | 76.9 |
| 25.33 | 3 | 11 | 0 | 0 | 0 | 3 | 100.0 | 72.7 | 100.0 | 70.5 |
| 31.11 | 3 | 8 | 0 | 1 | 0 | 4 | 100.0 | 46.7 | 100.0 | 51.3 |
| 36.89 | 3 | 3 | 0 | 1 | 0 | 0 | 100.0 | 100.0 | 100.0 | 23.9 |
| 42.67 | 3 | 2 | 0 | 0 | 2 | 1 | 33.3 | 50.0 | 100.0 | 23.9 |
| 48.44 | 1 | 1 | 0 | 0 | 0 | 1 | 100.0 | 0.0 | 33.3 | 12.0 |
| 54.22 | 1 | 0 | 0 | 0 | 0 | 0 | 100.0 | 0.0 | 33.3 | 0.0 |
| 60.00 | 1 | 0 | 0 | 0 | 1 | 0 | 0.0 | 0.0 | 33.3 | 0.0 |

TABLE 2
Life Table for Patients with TIGR Gene Mutation and At Least One
ApoE4 Allele, with Presence (T+) or Absence (T−) of an ApoE Gene
Promoter T Allele

| | | | No. | | No. with | | | | Cum. | |
|---|---|---|---|---|---|---|---|---|---|---|
| | No. Enter | | Censored | | Disease | | % Surviving | | % Surviving | |
| ApoE: | T− | T+ | T− | T+ | T− | T+ | T− | T+ | T− | T+ |
| Age | | | | | | | | | | |
| 10.00 | 13 | 4 | 2 | 0 | 1 | 2 | 91.7 | 50.0 | 100.0 | 100.0 |
| 14.44 | 10 | 2 | 1 | 0 | 1 | 0 | 89.5 | 100.0 | 91.7 | 50.0 |
| 18.89 | 8 | 2 | 1 | 0 | 0 | 0 | 100.0 | 100.0 | 82.0 | 50.0 |
| 23.33 | 7 | 2 | 1 | 0 | 0 | 2 | 100.0 | 0.0 | 82.0 | 50.0 |
| 27.78 | 6 | 0 | 0 | 0 | 0 | 0 | 100.0 | 0.0 | 82.0 | 0.0 |
| 32.22 | 6 | 0 | 1 | 0 | 3 | 0 | 45.5 | 0.0 | 82.0 | 0.0 |
| 36.67 | 2 | 0 | 0 | 0 | 0 | 0 | 100.0 | 0.0 | 37.3 | 0.0 |
| 41.11 | 2 | 0 | 0 | 0 | 1 | 0 | 50.0 | 0.0 | 37.3 | 0.0 |
| 45.56 | 1 | 0 | 0 | 0 | 0 | 0 | 100.0 | 0.0 | 18.6 | 0.0 |
| 50.00 | 1 | 0 | 0 | 0 | 1 | 0 | 0.0 | 0.0 | 18.6 | 0.0 |

In another assessment, a group of unrelated POAG patients was tested, regardless of family history of glaucoma and of linkage to TIGR, to determine whether ApoE alleles themselves influenced glaucoma phenotypes (age of onset and initial IOP). The allele frequencies of the ApoE gene were not significantly different in patients and in controls. The ApoE alleles did not influence glaucoma phenotype; however, the ApoE “T” gene promoter allele was associated with higher IOP at diagnosis (AA (n=85):31.4±8.3 vs AT+TT (n=29):26.6±10.1 (p=0.008).

This same promoter allele was also associated with higher IOP in patients carrying the TIGR gene promoter mutation mt1 (a G allele at position −1000 relative to the transcription start site of the TIGR gene, instead of a C allele) (P=0.005 for a comparison between Mt+ (positive)/ApoE-T and Mt−(negative)), as shown in Table 3.

TABLE 3
Higher IOP in mt1+/ApoE-T Patients

| | ApoE + T (“T” | | |
|---|---|---|---|
| Mt1 | allele of promoter) | IOP | N |
| N | N | 19.4 ± 5.6 | 75 |
| N | Y | 18.8 ± 5.5 | 25 |
| Y | N | 22.3 ± 8.1 | 19 |
| Y | Y | 29.0 ± 17.0 | 4 |

N = (−); Y = (+).

In addition, an interaction between the mt1 promoter mutation of TIGR and the ApoE alleles indicated that the ApoE4 allele was associated with a lower IOP at diagnosis (P=0.004):

TABLE 4
Lower Initial IOP in Mt1+/ApoE4 Patients

| Mt1 | ApoE4 allele | IOP | N |
|---|---|---|---|
| N | N | 31.8 ± 8.6 | 85 |
| N | Y | 36.8 ± 2.4 | 23 |
| Y | N | 37.5 ± 9.9 | 19 |
| Y | Y | 25.7 ± 4.8 | 4 |

N = (−); Y = (+).

### EXAMPLE 2

### Quantitative Association of ApoE Promoter SNPs With Intra-Ocular Pressure in Patients With POAG

A group of 191 unrelated POAG patients was identified through retrospective chart review, following a protocol previously described (Colomb et al., Clin. Genet. 60(3):220-225 (2001)). POAG was defined by the conjunction of a characteristic cupping of the optic disk, and open iridocorneal angle (grade III or IV gonioscopy) and an alteration of the visual field, tested by automated perimetry (with Humphrey's perimeter or Octopus). IOP was measured by applanation tonometry. Cup/disk ratios were assessed by individual clinicians. Patients with cataract or media opacities and those with a cause of secondary glaucoma, notably including exfoliation, pigment dispersion, history of trauma, surgery, and glucocorticoid exposure, were excluded. Final POAG diagnosis was made at the time of inclusion after review of inclusion and exclusion criteria. Diagnosis and other classifications were made substantially before genotyping was conducted. All patients had been checked for the absence of the TIGR/MYOC coding region mutations previously described in the French population, including G246R, Q368X, p370L, I477S, N480K, I499F and R272G (Adam et al., Hum. Mol. Genet. 6:2091-2097 (1997)).

Three informative SNPs in the ApoE promoter, at position −491, −427 and −219, were investigated along with the ApoE protein alleles E2, E3 and E4 (Fullerton et al., Am. J. Hum. Genet. 67:881-900 (2000)). Results are shown in Table 5.

TABLE 5
Frequencies of ApoE Promoter Genotypes

| ApoE Promoter | POAG Patients | Controls |
|---|---|---|
| SNP | n = 191 (%) | n = 102 (%) |
| −491 AA | 129 (67.5) | 70 (68.6) |
| −491 AT | 58 (30.4) | 28 (27.4) |
| −491 TT | 4 (2.1) | 4 (3.9) |
| −427 TT | 165 (86.4) | 84 (82.4) |
| −427 TC | 25 (13.1) | 17(16.7) |
| −427 TT | 1 (0.5) | 1 (0.98) |
| −219 GG | 57 (29.9) | 38 (37.2) |
| −219 GT | 105 (54.9) | 45 (44.1) |
| −219 TT | 29 (15.2) | 19 (18.6) |

As shown in Table 5, the genotype frequencies of the three SNPs were similar in POAG patients and in 102 controls who were healthy spouses from TIGR/MYOC-linked glaucoma families. Likewise, protein allele frequencies were similar in both groups. These frequencies were also similar to those reported in a larger group of French control persons (Lambert et al. Hum. Mol. Genet. 7:1511-1516 (1998)). These findings indicated that ApoE by itself was not a risk factor for POAG.

A modification of the principal clinical parameters of POAG by these ApoE polymorphisms was then examined. These parameters included the IOPs at diagnosis, when available, and at the time of inclusion in the study, and the cup/disk ratio and a visual field score as indices of optic nerve alteration. For the IOPs, and also for the age at diagnosis, the age at inclusion, the length of observation (period between age at inclusion and age at diagnosis), values were normally distributed as verified with the D statistic of Kolmogorov-Smirnov and with Shapiro-Wilk's W test. The means in genotypic groups were compared with the Student's t test or with one-way ANOVA. In case of variance heterogeneity, assessed with Levene's test, the Student's t test was performed after a separate estimation of variances. Because visual fields were not assessed in a standardized manner, a semi-quantitative grading procedure was used. Scores on a five-point scale were defined as previously (1, no alteration; 2, early defect; 3, arcuate defect; 4, advanced scotoma; 5, light perception only or no vision) (Brezin et al., J. Med. Genet. 34:546-552 (1997)). They were assigned prior to genotyping. Visual field scores and cup/disk ratios were compared with nonparametric procedures, including Mann-Whitney U-test and Kruskal-Wallis ANOVA test. In case of small samples, exact probabilities were determined. Only two-sided P values less than 0.05 were reported.

In the cases of ApoE (−491) and ApoE(−427), heterozygotes and homozygotes for the rarer allele were grouped. Results are shown in Table 6.

TABLE 7
Evolution of IOP Depending on MYOC.mt1 and on ApoE(−491T)

| | | | | | *Individual | ¶Relative | | P value of |
|---|---|---|---|---|---|---|---|---|
| | | | IOP at | IOP at | IOP | IOP | | the |
| | | | diagnosis | inclusion | variation | variation | §P value of | Wilcoxon |
| MYOC.mt | ApoE | | (mean ± std. | (mean ± std. | (mean ± std. | (mean ± std. | post-hoc | matched |
| 1 | (−491T) | N | dev.) mmHg | dev.) mmHg | dev.) mmHg | dev.) % | comparisons | pairs test |
| − | − | 89 | 31.4 ± 8.9 | 19.4 ± 4.6 | −12 ± 9.8 | −34.3 ± 21 | 3 × 10⁵ | <1 × 10⁻¹² |
| − | + | 34 | 35.1 ± 10.2 | 18.7 ± 4.6 | −16.4 ± 1.5 | −42.5 ± 20.2 | 3 × 10⁵ | 1 × 10⁻⁶ |
| + | − | 20 | 32 ± 10.1 | 21 ± 7.2 | −10.9 ± 13.5 | −27.9 ± 31.4 | 3 × 10⁴ | 3 × 10⁻³ |
| + | + | 5 | 35.4 ± 7.1 | 35.2 ± 7.9 | −0.2 ± 8.2 | −0.7 ± 21.7 | NS | NS |

*Individual difference between IOP at inclusion in the study and IOP at diagnosis
¶Individual IOP variation divided by IOP at diagnosis
§Between IOP at diagnosis and IOP at inclusion in the study, with Tukey honest significant difference test following three-factor ANOVA

As seen in Table 6, the ApoE (−491T) allele was associated with a higher IOP at diagnosis (P=0.01) whereas the ApoE (−219) SNP influenced both the visual field score (P=0.003) and the cup/disk ratio (P=0.04). The means of the latter two variables were similar in patients carrying one or two G alleles and higher than in TT homozygotes. Grouping GG and GT patients improved fitting the data (P=0.0012 for VF score and P=0.014 for c/d ratio). In contrast, the ApoE (−427) SNP and the E2/E3/E4 allele system did not affect the parameters listed in Table 6.

It was previously shown that an allele of a SNP in the promoter of the TIGR/MYOC gene (−1000G), also designated as MYOC.mt1, was associated with higher IOP at inclusion in the study and with a lack of IOP lowering between diagnosis and the time of the study (Colomb et al., supra). The effect of MYOC.mt1 on individual changes in IOP in the period from diagnosis to inclusion in the study was reevaluated, also taking into account the influence of ApoE(−491T). This analysis was carried out with a three-factor ANOVA, including a repetitive measures factor (“within-subjects” variation of IOP) and MYOC.mt1 and ApoE(−491T) as independent (“between-groups”) factors. Individual variations in IOP between diagnosis and inclusion in the study were normally distributed and there was no correlation between these two variables. In the whole group of patients, the individual variation of IOP was highly significant (P=1×10⁻¹²), reflecting efficient IOP lowering in treated patients. The effect of both genetic markers was also significant (P 0.001 for MYOC.mt1 and P=0.0004 for ApoE(−491T )). Moreover, there was an interaction between them (P=0.012). Post-hoc comparisons of IOP means with Tukey honest significant difference test indicated that within-subjects variation of IOP was significant in all patient groups except in those positive for both MYOC.mt1 and ApoE(−491T), as shown in Table 7, below, and confirmed by the nonparametric Wilcoxon matched pairs test.

TABLE 6
Association of ApoE Promoter SNPs with POAG Phenotypes

| | ApoE(−491) Genotype | | ApoE(−219) Genotype | | |
|---|---|---|---|---|---|
| | AA | AT or TT | GG | GT | TT |
| Age at Diagnosis (yrs) | 56.9 ± 15.6 (127) | 60.6 ± 15.2 (62) | 59.4 ± 15.6 (56) | 57.2 ± 15.8 (105) | 58.5 ± 14.8 (28) |
| Age at inclusion (yrs) | 45 ± 13.6 (123) | 47.2 ± 16.5 (58) | 48.1 ± 12.6 (54) | 44.5 ± 15.4 (100) | 45.4 ± 14.7 (27) |
| Length of observation* (yrs) | 12.6 ± 8.2 (122) | 13.6 ± 9 (58) | 12.9 ± 7.2 (53) | 12.8 ± 8.4 (100) | 13.4 ± 11.1 (27) |
| IOP at diagnosis (mmHg) | 31.3 ± 9# (112) | 35.6 ± 10.1# (43) | 30.9 ± 7.5 (48) | 33.3 ± 10.4 (86) | 33 ± 9.6 (21) |
| IOP at inclusion (mmHg) | 19.6 ± 5.4 (125) | 20 ± 6.6 (56) | 19.9 ± 5.8 (56) | 19.3 ± 5.9 (97) | 20.9 ± 5.1 (28) |
| Visual field score | 3.2 ± 1 (118) | 3.2 ± 1 (51) | ^{3.2 ± 0.8¶ (52)} | ^{3.3 ± 1.1¶ (93)} | ^{2.6 ± 0.9¶ (24)} |
| Cup/disk ratio (× 10) | 7.3 ± 2.1 (112) | 7.3 ± 1.7 (51) | 7.6 ± 1.8§ (51) | 7.3 ± 2.1§ (86) | 6.6 ± 1.7§ (26) |

*Between diagnosis and inclusion in the study
#P = 0.01
¶P = 0.003 for the comparison of the three genotypic groups; P = 0.0012 for comparison of GG or GT versus TT.
§P = 0.04 for the comparison of the three genotypic groups; P = 0.014 for comparison of GG or GT versus TT.

Thus, patients carrying both MYOC.mt1 and ApoE(−491T) failed to decrease their IOP significantly with the treatment protocols used between diagnosis and the time of the study. Their IOP level remained well above 20 mmHg. In contrast, the decrease of IOP in patients that did not carry the MYOC.mt1 polymorphism was very significant and greater then 30%, resulting in levels in average lower than 20 mmHg. Interestingly, subjects who carried only MYOC.mt1 but not ApoE(−491T) also seemed to be less responsive to therapy. Their relative decrease in IOP was less pronounced compared to the whole group of patients negative for MYOC.mt1 (P=0.04). A two-factor ANOVA of individual IOP variation and of relative IOP variation, indicated that they were influenced by NYOC.mt1 and by a combination of MYOC.mt1 with ApoE(−491T), as shown in Table 8.

TABLE 8
Covariance analysis and Interaction of MYOC.mt1 and ApoE(−491T)

| | Individual | | | | | |
|---|---|---|---|---|---|---|
| | IOP Variation | | Relative IOP Variation | | | |
| Variable | | IOP at | IOP | | IOP at | IOP |
| Covariate | None | Diagnosis | Mean | None | Diagnosis | Mean |
| MYOC.mt1 | 0.003 | 1 × 10⁻⁹ | 2 × 10⁻⁷ | 7 × 10−5 | 7 × 10⁻⁸ | 2 × 10⁻⁶ |
| | * | | | | | |
| ApoE | NS | 4 × 10⁻⁶ | 9 × 10⁻⁴ | NS | 5 × 10⁻⁴ | 1 × 10⁻² |
| (491T) | | | | | | |
| Interaction | 0.01 | 4 × 10⁻⁷ | 1 × 10⁻⁵ | 0.003 | 9 × 10⁻⁵ | 3 × 10⁻⁴ |
| term | | | | | | |

* P values for each factor and for their interaction in a two-way ANOVA on individual IOP variation (IOP at inclusion in the study minus IOP at diagnosis) or on relative TOP variation (IOP variation//IOP at diagnosis or initial IOP).

When the IOP at diagnosis was introduced as a covariate, an effect of ApoE(−491T) was detected and the significance levels of MYOC.mt1 and of its interaction with ApoE(−491T) were considerably increased.

These findings extend and refine the previous observation of an association of MYOC.mt1 with resistance to IOP control and strengthen the theory of a genetic basis for resistance. The association of ApoE(−219) SNP with cup/disk ratio and visual field score suggests for the first time that these two parameters, which are commonly monitored to assess optic nerve damage in POAG patients, are influenced by genetic factors independently of IOP. At the molecular level, both −219 and −491 SNP, but not −427 SNP, alter transcriptional activity of ApoE as shown by transient transfection and electrophoretic mobility shift assays (Artiga et al., Febs. Lett. 421:105-108 (1998)). The different effect of these two SNPs on IOP and on optic nerve damage provides a genetic basis for the discrepancies between IOP level and optic nerve damage in POAG patients and in ocular hypertensive subjects. A modification of POAG phenotype by ApoE is consistent with the recent implication of inflammatory signaling pathway in IOP increase at an early stage of POAG (Wang et al., Nat. Med. 7:304-309 (2001)).

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. A method of assessing the risk of an individual for developing early-onset glaucoma, comprising assessing the ApoE alleles in the individual, wherein if the individual is a carrier of a TIGR gene mutation, the presence of an ApoE4 allele is indicative of an increased risk of developing early-onset glaucoma, and the absence of any ApoE4 alleles is indicative of a decreased risk of developing early-onset glaucoma.

2. The method of claim 1, wherein assessing the ApoE alleles in the individual comprises detecting the presence or absence of an ApoE4 allele in the individual.

3. The method of claim 1, further comprising detecting the presence or absence of a TIGR gene mutation in the individual.

4. A method of assessing the risk of an individual for developing glaucoma with a high intraocular pressure at onset of disease, comprising assessing the ApoE alleles in the individual, wherein if the individual is a carrier of a TIGR gene promoter mutation, the presence of an ApoE4 allele is indicative of a decreased risk of developing glaucoma with a high intraocular pressure at onset of disease, and the absence any ApoE4 alleles is indicative of an increased risk of developing glaucoma with a high intraocular pressure at onset of disease.

5. The method of claim 4, wherein assessing the ApoE alleles in the individual comprises detecting the presence or absence of an ApoE4 allele in the individual.

6. The method of claim 4, further comprising detecting the presence or absence of a TIGR gene mutation in the individual.

7. A method of assessing the risk of an individual for developing early-onset glaucoma, comprising assessing the alleles of the ApoE gene promoter in the individual, wherein if the individual is a carrier of a TIGR gene mutation and has an ApoE4 allele, the presence of a T allele in an ApoE gene promoter is indicative of an increased risk of developing early-onset glaucoma, and the absence of any T alleles in an ApoE gene promoter is indicative of a decreased risk of developing early-onset glaucoma.

8. The method of claim 7, wherein assessing the alleles of the ApoE gene promoter in the individual comprises detecting the presence or absence of a T allele of the ApoE gene promoter in the individual.

9. The method of claim 7, further comprising detecting the presence or absence of a TIGR gene mutation in the individual and detecting the presence or absence of an ApoE4 allele in the individual.

10. A method of assessing the risk of an individual for developing glaucoma with a high intraocular pressure at onset of disease, comprising assessing the alleles of the ApoE gene promoter in the individual, wherein the presence of a T allele in an ApoE gene promoter is indicative of an increased risk of developing glaucoma with a high intraocular pressure at onset of disease, and the absence of any T alleles in an ApoE gene promoter is indicative of a decreased risk of developing glaucoma with a high intraocular pressure at onset of disease.

11. The method of claim 10, wherein assessing the alleles of the ApoE gene promoter in the individual comprises detecting the presence or absence of a T allele of the ApoE gene promoter in the individual.

12. The method of claim 10, wherein the individual is a carrier of a TIGR gene mutation.

13. The method of claim 12, wherein the presence of any T alleles in an ApoE gene promoter is indicative of an even more increased risk of developing glaucoma with a high intraocular pressure at onset of disease.

14. The method of claim 13, wherein the TIGR gene promoter mutation is (−1000G).

15. A method of assessing the risk of an individual for developing glaucoma with a high visual field score, a high cup/disk ratio, or both a high visual field score and a high cup/disk ratio, comprising assessing the ApoE gene promoter alleles in the individual, wherein the presence of an ApoE(−219G) gene promoter allele is indicative of an increased risk of developing glaucoma with a high visual field score, high cup/disk ratio, or both a high visual field score and a high cup/disk ratio, and the absence an ApoE(−219G) gene promoter allele is indicative of a decreased risk of developing glaucoma with a high visual field score or high cup/disk ratio.

16. A kit for determining whether an individual is at increased risk or decreased risk of developing early-onset glaucoma, comprising:
a) at least one reagent that can be used to detect the presence or absence of an ApoE4 allele in the individual, and
b) instructions for determining whether the individual is at increased risk or decreased risk of developing early-onset glaucoma, by determining the presence or absence of an ApoE4 allele, wherein if the individual is a TIGR gene mutation carrier, the presence of an ApoE4 allele is indicative of an increased risk of developing early-onset glaucoma, and the absence of any ApoE4 alleles is indicative of a decreased risk of developing early-onset glaucoma.

17. A kit for determining whether an individual is at increased risk or decreased risk of developing glaucoma with a high intraocular pressure at onset of disease, comprising:
a) at least one reagent that can be used to detect the presence or absence of an ApoE4 allele in the individual, and instructions for determining whether the individual is at increased risk or decreased risk of developing glaucoma with a high intraocular pressure at onset of disease, by determining the presence or absence of an ApoE4 allele, wherein if the individual is a TIGR gene promoter mutation carrier, the presence of an ApoE4 allele is indicative of a decreased risk of developing glaucoma with a high intraocular pressure at onset of disease, and the absence of any ApoE4 alleles is indicative of an increased risk of developing glaucoma with a high intraocular pressure at onset of disease; or
b) at least one reagent that can be used to detect the presence or absence of an T allele in the ApoE gene promoter in the individual, and instructions for determining whether the individual is at increased risk or decreased risk of developing glaucoma with a high intraocular pressure at onset of disease, by determining the presence or absence of a T allele in the ApoE gene promoter, wherein the presence of a T allele in the ApoE gene promoter is indicative of an increased risk of developing glaucoma with a high intraocular pressure at onset of disease, and the absence of any T alleles of the ApoE gene promoter is indicative of a decreased risk of developing glaucoma with a high intraocular pressure at onset of disease; or
c) both (a) and (b).

18. A kit for determining whether an individual is at increased risk or decreased risk of developing glaucoma with a high intraocular pressure at onset of disease, comprising:
a) at least one reagent that can be used to detect the presence or absence of an ApoE(−491T) gene promoter allele in the individual, and
b) instructions for determining whether the individual is at increased risk or decreased risk of developing glaucoma with a high intraocular pressure at onset of disease, by determining the presence or absence of an ApoE(−491T) gene promoter allele, wherein the presence of an ApoE(−491T) gene promoter allele is indicative of an increased risk of developing glaucoma with a high intraocular pressure at onset of disease, and the absence of any ApoE(−491T) gene promoter alleles is indicative of a decreased risk of developing glaucoma with a high intraocular pressure at onset of disease.

19. A kit for determining whether an individual is at increased risk or decreased risk of developing glaucoma with a high visual field score, a high cup/disk ratio, or both, comprising:
a) at least one reagent that can be used to detect the presence or absence of an ApoE(−219G) gene promoter allele in the individual, and
b) instructions for determining whether the individual is at increased risk or decreased risk of developing glaucoma with a high visual field score, a high cup/disk ratio, by determining the presence or absence of an ApoE(−219G) gene promoter allele, wherein the presence of an ApoE(−219G) gene promoter allele is indicative of an increased risk of developing glaucoma with a high visual field score and/or a high cup/disk ratio, and the absence of any ApoE(−219G) gene promoter alleles is indicative of a decreased risk of developing glaucoma with a high visual field score or a high cup/disk ratio.
